(19) United States
(12) Patent Application Publication
(10) Pub. No.: US 2006/0173241 A1
(43) Pub. Date: Aug. 3, 2006

(54) Front end structure of endoscope

(75) Inventors: Teruo Ouchi, Saitama (JP); Makoto Kobayashi, Mie (JP)

Correspondence Address: GREENBLUM & BERNSTEIN, P.L.C., 1950 ROLAND CLARKE PLACE, RESTON, VA, 20191, US

(73) Assignee: PENTAX Corporation, Tokyo (JP)

(21) Appl. No.: 11/331,168

(22) Filed: Jan. 13, 2006

(30) Foreign Application Priority Data
Jan. 14, 2005 (JP) 2005-006975
Jan. 14, 2005 (JP) 2005-006976
Jan. 14, 2005 (JP) 2005-006977
Jan. 14, 2005 (JP) 2005-006978
Jan. 14, 2005 (JP) 2005-006980

(51) Int. Cl. A61B 1/00; A61B 1/04

(52) U.S. Cl. 600/129; 600/128; 600/127

(57) ABSTRACT

An endoscope having an inserted portion, which is to be inserted into a tubular organ, including a bendable portion that is adapted to bend arbitrarily by a remote operation and a distal unit with a window for observation to capture an image of inside of the tubular organ, is provided. At least one protrusion is formed on a outer peripheral surface of the bendable portion.

### BACKGROUND OF THE INVENTION

The present invention relates to an endoscope to be inserted into a tubular organ including a large intestine.

When an endoscope is inserted into a tubular organ such as a large intestine through an anus, an inserted portion of the endoscope proceeds in the winding intestine through a relatively short rectum, an S-shaped sigmoid colon with a small curvature, a descending colon and a traverse colon until the inserted portion reaches to an ascending colon. Specifically, a part of the intestine between the descending colon and the traverse colon is bent at an angle that is generally sharper than the right angle, therefore it has been difficult for operators to insert the endoscope deeply in the colons.

In addition, most part of the colons is movable in a body cavity and not held to a body wall, thus the colons, specifically the sigmoid colon, are easily elongated, shortened or deformed by external force. Therefore, even if the endoscope is forcibly inserted into the colons, the colons are merely elongated by the inserted portion, and the inserted portion is not effectively advanced deeply in the colons.

In consideration of the above problems, when the inserted portion is inserted in the colons and advanced to an extent, a bendable portion of the endoscope, which is configured at a distal end of the inserted portion, is bent at a small curvature by a remote operation from the operator, and the distal end is pressed and hooked to a creased portion of an inner wall of the colon. With the distal end hooked to the crease, the inserted portion is pulled toward the anus, thus the colon is partially folded up as an accordion and shortened, or the inserted portion is twisted in a circumferential direction along with the inserted portion, thus the colon is turned to be partially straightened, so that the inserted portion can be advanced further with respect to the colon.

The bendable portion of such an endoscope is generally coated with a flexible tubular member. An example of such an endoscope is disclosed in Japanese Patent Provisional Publication HEI9-24020.

Further, an endoscope for a large intestine with the inserted portion and the bendable portion is generally provided with an observation window at the distal end for capturing an image of an object, which is ahead of the inserted portion. In such case, the inserted portion is often provided further with a hood that protrudes from an outer edge of the inserted portion to prevent the observation window from directly being in contact with an intestinal wall. An example of such an endoscope is disclosed in Japanese Patent Provisional Publication HEI10-248792. In recent years, there are also provided with endoscopes having observation windows and no hoods. An example of such an endoscope is disclosed in Japanese Patent Provisional Publication 2002-125915.

When an endoscope as described above is inserted into the colons and pulled or twisted to partially straighten the colon, the bendable portion hooked to the crease may slip and be unhooked from the crease unintentionally. In such case, the operator is required to repeat the hooking and pulling or twisting operations, which consume time and may be troublesome.

### SUMMARY OF THE INVENTION

Aspects of the present invention are advantageous in that an endoscope having an inserted portion, which is to be inserted into a tubular organ, including a bendable portion that is adapted to bend arbitrarily by a remote operation and a distal unit with a window for observation to capture an image of inside of the tubular organ, is provided. At least one protrusion is formed on a outer peripheral surface of the bendable portion.

Optionally, the bendable portion may be coated with a flexible tubular member, and the at least one protrusion may be integrally formed with the tubular member.

Optionally, the at least one protrusion may be protrusively formed in parallel with a circumferential direction of the bendable portion.

Optionally, a plurality of the at least one protrusion may be spaced from each other in an axial direction of the bendable portion.

Optionally, the at least one protrusion may be protrusively formed in parallel with an axial direction of the bendable portion.

Optionally a plurality of the at least one protrusion may be spaced from each other in parallel with a circumferential direction of the bendable portion.

Optionally, at least one protrusion is formed in parallel with an axial direction of the bendable portion, and at least one protrusion may be formed in parallel with a circumferential direction of the bendable portion.

Optionally, the at least one protrusion may be formed to be in a line of a spiral.

Optionally, a plurality of the at least one protrusion may be formed to be in a broken line.

Optionally, the at least one protrusion may be formed in a vicinity of a distal end of the bendable portion.

Optionally, the at least one protrusion may be formed on an entire length of the bendable portion.

Aspects of the present invention are advantageous in that an endoscope having an inserted portion, which is to be inserted into a tubular organ, including a bendable portion that is adapted to bend arbitrarily by a remote operation and a distal unit with a window for observation to capture an image of inside of the tubular organ, is provided. At least one flange with an outer diameter being greater than an outer diameter of the bendable portion is protrusively formed on an outer peripheral surface of the distal unit.

Optionally, a plurality of the at least one flange may be spaced from each other on the outer peripheral surface of the distal unit.

Optionally, one of the plurality of the at least one flange that may be closer to a distal end of the distal unit is formed to have an outer diameter that is smaller than an outer diameter of another one of the plurality of the at least one flange that is closer to a proximal end of the distal unit.

Optionally, a plurality of recessed portions may be formed on the at least one flange and spaced from each other in parallel with a circumferential direction of the at least one flange.

Optionally, the at least one flange may be formed to be tapered toward the distal end of the distal unit.

Aspects of the present invention are advantageous in that an endoscope having an inserted portion, which is to be inserted into a tubular organ, including a bendable portion that is adapted to bend arbitrarily by a remote operation and a distal unit with a window for observation to capture an image of inside of the tubular organ, and a cylindrical hood that is provided to a distal end of the distal unit and formed to protrude forwardly from the distal end of the distal unit, is provided. At least one flange that encircles the cylindrical hood in a circumferential direction of the cylindrical hood is attached to an outer peripheral surface of the cylindrical hood.

Optionally, the cylindrical hood may be made of a transparent material.

Optionally, the cylindrical hood may be configured to be detachable from the distal unit.

Optionally, the at least one flange may be formed integrally with the cylindrical hood.

Optionally, the at least one flange may include a proximal edge that is closer to a proximal end of the cylindrical hood and a distal edge that is closer to a distal end of the cylindrical hood, the distal edge being chamfered in a greater curvature than a curvature of the proximal edge being chamfered.

Optionally, the cylindrical hood may include a tapered portion that is tapered from a position where the at least one flange is fixed to toward a distal end of the cylindrical hood.

Optionally, an outer peripheral surface of the at least one hood may be formed to be tapered toward a distal end of the cylindrical hood.

Aspects of the present invention are advantageous in that an endoscope having an inserted portion, which is to be inserted into a tubular organ, including a bendable portion that is adapted to bend arbitrarily by a remote operation and a distal unit with a window for observation to capture an image of inside of the tubular organ, is provided. The distal unit is detachably attached with a support band that encircles the distal unit in parallel with a circumferential direction of the distal unit. The support band is integrally formed with at least one flange that protrudes outwardly and annularly in parallel with the circumferential direction of the support band.

Optionally, the support band may be configured to be attached to the distal unit so that the support band remain in an attached position.

Optionally, the support band may be attached to the distal unit by being elastically tightened around an outer peripheral surface of the distal unit.

Optionally, the at least one flange may be formed to be completely included in the support band.

Optionally, the at least one flange may be formed to be tapered toward a distal end of the distal unit.

Optionally, a plurality of the at least one flange may be formed to be spaced from each other in parallel with a circumferential direction of the support band.

Optionally, one of the plurality of the at least one flange that may be closer to a proximal end of the distal unit is formed to be tapered toward the proximal end of the distal unit.

Optionally, a maximum outer diameter of one of the plurality of the at least one flange that is closer to a distal end of the distal unit may be formed to be smaller than a maximum outer diameter of the one of the plurality of the at least one flange that is closer to the proximal end of the distal unit.

Optionally, a plurality of recessed portions may be formed on the at least one flange and spaced from each other in parallel with a circumferential direction of the at least one flange.

Aspects of the present invention are advantageous in that an endoscope having an inserted portion, which is to be inserted into a tubular organ, including a bendable portion that is adapted to bend arbitrarily by a remote operation and a distal unit with a window for observation to capture an image of inside of the tubular organ, and a cylindrical hood that is provided to a distal end of the distal unit and formed to protrude forwardly from the distal end of the distal unit, is provided. A plurality of bosses having a rear surface and a peripheral surface that protrude outwardly are formed on an outer peripheral surface of the cylindrical hood and spaced from each other.

Optionally, the cylindrical hood may be made of a transparent material.

Optionally, the cylindrical hood may be configured to be detachable from the distal unit.

Optionally, the plurality of bosses may be formed integrally with the cylindrical hood.

Optionally, each of the plurality of bosses may include a tapered portion that is tapered from a position where the each of the plurality of bosses is most protruded outwardly toward a distal end of the cylindrical hood.

Optionally, an outer peripheral surface of the plurality of bosses may be formed to be tapered toward a distal end of the cylindrical hood.

### BRIEF DESCRIPTION OF THE ACCOMPANYING DRAWINGS

FIG. 1 is an external view of an entire configuration of an endoscope according to the first embodiment of the present invention.

FIG. 2 is a side view of the inserted portion of the endoscope according to the first embodiment of the present invention.

FIG. 3 is an illustrative view of the endoscope in use according to the first embodiment of the present invention.

FIG. 4 is another illustrative view of the endoscope in use according to the first embodiment of the present invention.

FIG. 5 is a side view of an inserted portion of an endoscope according to a second embodiment of the present invention.

FIG. 6 is a cross-sectional view of a distal unit of the endoscope taken along the line VI-VI in FIG. 5.

FIG. 7 is an illustrative view of the endoscope in use according to the second embodiment of the present invention.

FIG. 8 is a side view of an inserted portion of an endoscope according to a third embodiment of the present invention.

FIG. 9 is a side view of an inserted portion of an endoscope according to a fourth embodiment of the present invention.

FIG. 10 is a side view of an inserted portion of an endoscope according to a fifth embodiment of the present invention.

FIG. 11 is a side view of an inserted portion of an endoscope according to a sixth embodiment of the present invention.

FIG. 12 is a side view of an inserted portion of an endoscope according to a seventh embodiment of the present invention.

FIG. 13 is a side view of an inserted portion of an endoscope according to an eighth embodiment of the present invention.

FIG. 14 is a side view of an inserted portion of an endoscope according to a ninth embodiment of the present invention.

FIG. 15 is a side view of an inserted portion of an endoscope according to a tenth embodiment of the present invention.

FIG. 16 is a side view of an inserted portion of an endoscope according to an eleventh embodiment of the present invention.

FIG. 17 is a cross-sectional view of a flange taken along the line XVII-XVII in FIG. 16.

FIG. 18 is a side view of an inserted portion of an endoscope according to a twelfth embodiment of the present invention.

FIG. 19 is a cross-sectional side view of an inserted portion of an endoscope according to a thirteenth embodiment of the present invention.

FIG. 20 is a perspective view of a flange of the endoscope according to the thirteenth embodiment of the present invention.

FIG. 21 is a cross-sectional side view of an inserted portion of an endoscope according to a fourteenth embodiment of the present invention.

FIG. 22 is a cross-sectional side view of an inserted portion of an endoscope according to a fifteenth embodiment of the present invention.

FIG. 23 is a cross-sectional side view of an inserted portion of an endoscope according to a sixteenth embodiment of the present invention.

FIG. 24 is a cross-sectional side view of an inserted portion of an endoscope according to a seventeenth embodiment of the present invention.

FIG. 25 is a cross-sectional side view of an inserted portion of an endoscope according to an eighteenth embodiment of the present invention.

FIG. 26 is a side view of an inserted portion of an endoscope according to a nineteenth embodiment of the present invention.

FIG. 27 is a perspective view of a support band of the endoscope according to the nineteenth embodiment of the present invention.

FIG. 28 is a side view of an inserted portion of an endoscope according to a twentieth embodiment of the present invention.

FIG. 29 is a side view of an inserted portion of an endoscope according to a twenty-first embodiment of the present invention.

FIG. 30 is a side view of an inserted portion of an endoscope according to a twenty-second embodiment of the present invention.

FIG. 31 is a side view of an inserted portion of an endoscope according to a twenty-third embodiment of the present invention.

FIG. 32 is a side view of an inserted portion of an endoscope according to a twenty-fourth embodiment of the present invention.

FIG. 33 is a cross-sectional view of a support band with a flange of an endoscope taken along a line that is perpendicular to an axial direction of the support band according to a twenty-fifth embodiment of the present invention.

FIG. 34 is a side view of an inserted portion of an endoscope according to a twenty-sixth embodiment of the present invention.

FIG. 35 is a perspective side view of the inserted portion of the endoscope according to the twenty-sixth embodiment of the present invention.

FIG. 36 is a cross-sectional view of a hood of the endoscope according to the twenty-sixth embodiment of the present invention taken along the line XXXVI-XXXVI in FIG. 34.

FIG. 37 is another example of a cross-sectional view of the hood of the endoscope according to the twenty-sixth embodiment of the present invention taken along the line XXXVI-XXXVI in FIG. 34.

FIG. 38 is a cross-sectional side view of an inserted portion of an endoscope according to a twenty-seventh embodiment of the present invention.

FIG. 39 is a cross-sectional side view of an inserted portion of an endoscope according to a twenty-eighth embodiment of the present invention.

FIG. 40 is a cross-sectional side view of an inserted portion of an endoscope according to a twenty-ninth embodiment of the present invention.

FIG. 41 is a cross-sectional side view of an inserted portion of an endoscope according to a thirtieth embodiment of the present invention.

### DETAILED DESCRIPTION OF THE EMBODIMENT

Referring to the accompanying drawings, an endoscope according to an embodiment of the invention will be described in detail.

### First Embodiment

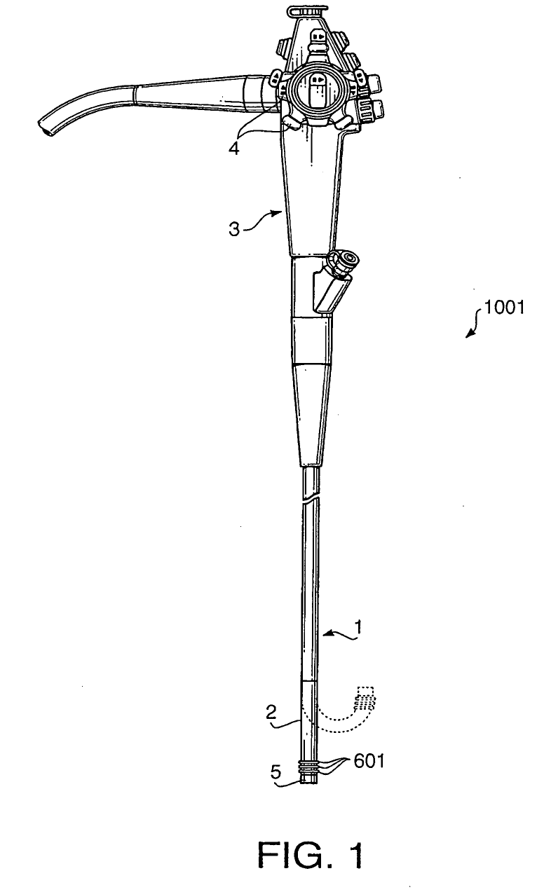

FIG. 1 is an external view of an entire configuration of an endoscope 1001 according to a first embodiment of the present invention. The endoscope 1001 is provided with an inserted portion 1. The inserted portion 1 includes a bendable portion 2, which is coated with a flexible coating member, in a vicinity to a distal end of the endoscope 1001. The endoscope 1001 is further provided with an operation unit 3 that is attached to the proximal end of the inserted portion 1 and operation knobs 4. The bendable portion 2 is configured to bend at an arbitrary curvature in an arbitrary direction, for example as shown in the dotted lines in FIG. 1, by an operation to the operation knobs 4.

At the distal end of the endoscope 1001, the bendable portion 2 is provided with an inflexible and short column-like distal unit 5, in which various functional components for example an optical system and a solid-state image capturing element are installed. On an outer peripheral surface of the bendable portion 2, a plurality of annular protrusions 601, which are spaced from each other, are protrusively formed in parallel with a circumferential direction of the bendable portion 2.

The bendable portion 2 is in a known configuration, including a plurality of ring members, which are sequentially connected with rivets so as to rotate about the rivets with respect to each other, to be bendable in an arbitrary direction by an operation to an operation wire. The operation wire is configured to bend the bendable portion by being drawn toward the operation knobs 4. The outer surface of the bendable portion 2 is coated with, for example, an elastic tube made of rubber.

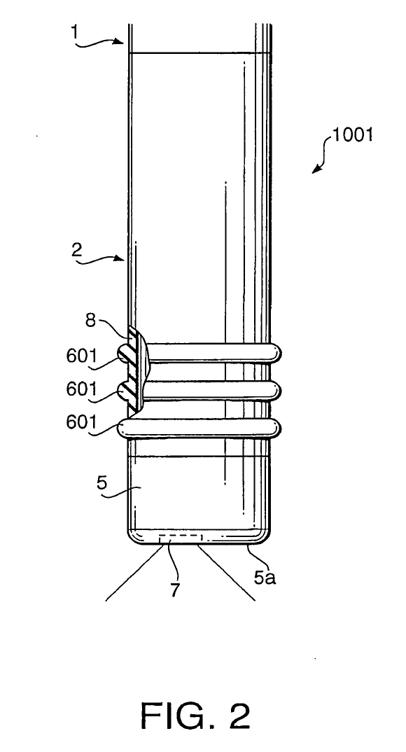

FIG. 2 is a side view of the inserted portion 1 with the bendable portion 2 and the distal unit 5 of the endoscope 1001 according to the first embodiment of the present invention, and On a distal surface 5a of the distal unit 5, a plurality of windows including an observation window 7 and an illumination window (not shown) to capture an image of an object are provided, so that the operator can observe inside of the colons.

The protrusions 601 are formed integrally with a flexible tubular member 8 that covers the bendable portion 2. Each of the protrusions 601 is formed to encircle the circumferential surface of the bendable portion 2, protruding outwardly with a cross-sectional shape of an arc.

In the present embodiment, the protrusions 601 are evenly spaced to each other, and arranged in a line perpendicularly to an axial direction of the bendable portion 2. It should be noted, however, the protrusions 601 are not necessarily spaced evenly to each other. Further, the number of the protrusion 601 is not limited to three, as long as at least one protrusion 601 is provided to the bendable portion 2.

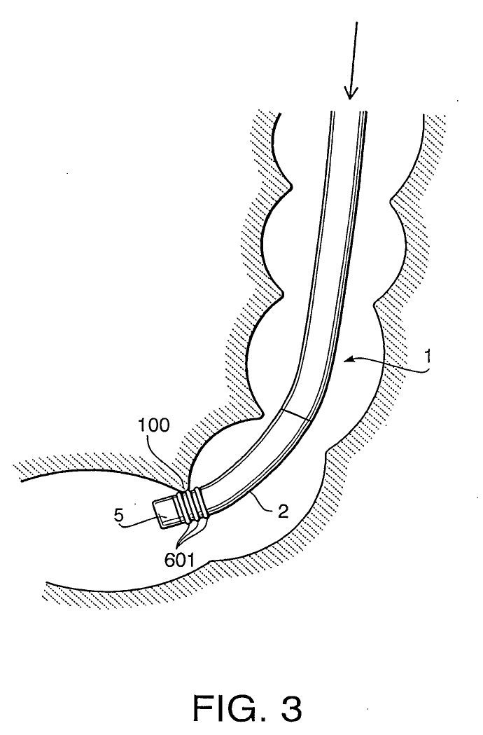

FIG. 3 is an illustrative view of the endoscope 1001 in use according to the first embodiment of the present invention. The bendable portion 2 with the distal unit 5 that passed by a crease 100 is bent by a remote operation, and the protrusions 601 of the distal unit 5 are pressed and hooked to the crease 100.

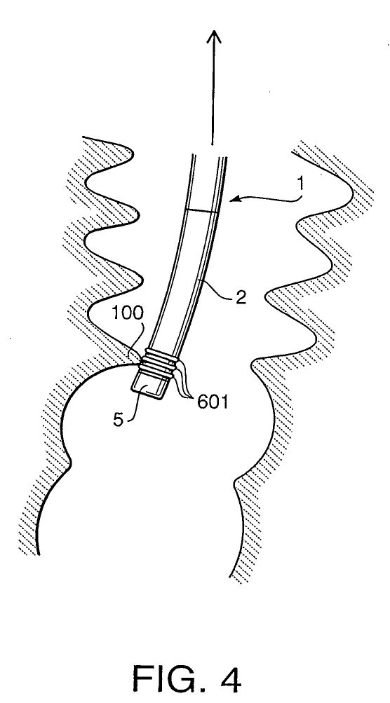

FIG. 4 is another illustrative view of the endoscope 1001 in use according to the first embodiment of the present invention. With the protrusions 601 hooked to the crease 100, when the inserted portion 1 is pulled for example in a direction indicated by an arrow in FIG. 4 toward the anus, the colon toward the anus is partially shortened, and the trailed part of the colon is straightened. The colon may be succeedingly twisted in the circumferential direction of the inserted portion, so that the looped part of the colon is turned along with the inserted portion 1 to be partially straightened. Thus the inserted portion 1 can be further advanced with respect to the colon.

In the above-described operations, the distal unit 5 with the protrusions 601 of the endoscope of the present invention is firmly hooked to the crease 100, and the bendable portion 2 is less likely to be detached unintentionally from the crease 100. Therefore, the colons are effectively shortened and straightened, and the inserted portion 1 can be securely advanced in the colons.

It should be noted that the configuration of the protrusions 601 is not limited to annular, but may be in various forms. For example, at least one of the protrusions 601 may be formed in a corrugated annularity, in parallel with the circumferential direction of the bendable portion 2. Further, protrusions of a plurality of forms may be formed in combination on the circumferential surface of the bendable portion 2.

### Second Embodiment

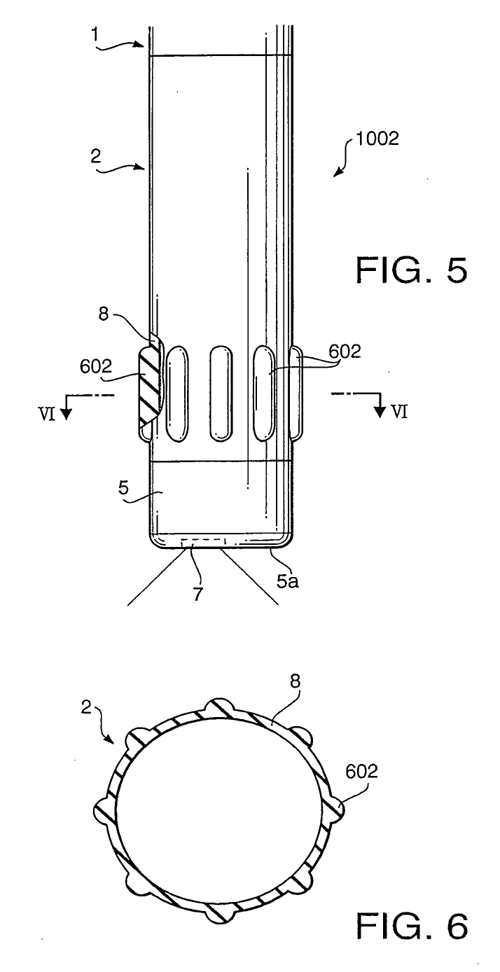

FIG. 5 is a side view of an inserted portion of an endoscope 1002 according to a second embodiment of the present invention. FIG. 6 is a cross-sectional view of a bendable portion 2 of the endoscope taken along the line VI-VI in FIG. 5. In this and the following embodiments, configurations corresponding to the configuration of the first embodiment is referred to by the same reference numbers, and description of those is omitted.

In the present embodiment, eight axial protrusions 602, which are evenly spaced from each other, are protrusively formed in parallel with the axial direction of the bendable portion 2 when the bendable portion 2 is linearly straightened. It should be noted, however, the protrusions 602 are not necessarily spaced evenly to each other. Further, the number of the protrusion 602 is not limited to eight, as long as at least one protrusion 602 is provided to the bendable portion 2, although it is preferable that the number of the protrusions 602 is in a range from three to twelve for practical reasons.

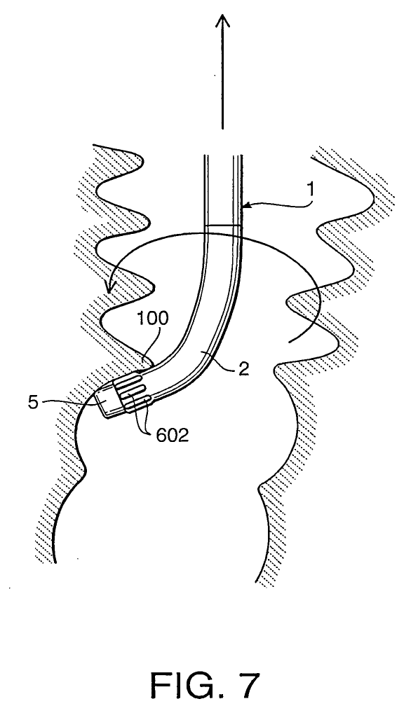

With the above-described configuration, the protrusions 602 can be hooked effectively to the crease 100. FIG. 7 is an illustrative view of the endoscope 1002 in use according to the second embodiment of the present invention. With the protrusions 602 hooked to the crease 100, when the inserted portion 1 is twisted in the circumferential direction, for example as indicated by an arrow in FIG. 7, the looped colon is turned along with the inserted portion 1 to be partially straightened. In addition, the crease 100 hooked with the protrusions 602 can be pulled toward the anus, thus the colon toward the anus can be partially shortened, and the trailed part of the colon is straightened, so that the inserted portion 1 can be further advanced with respect to the colon.

It should be noted that at least one of the protrusions 602 may be formed, for example, to be corrugated, in parallel with the axial direction of the bendable portion 2.

### Third Embodiment

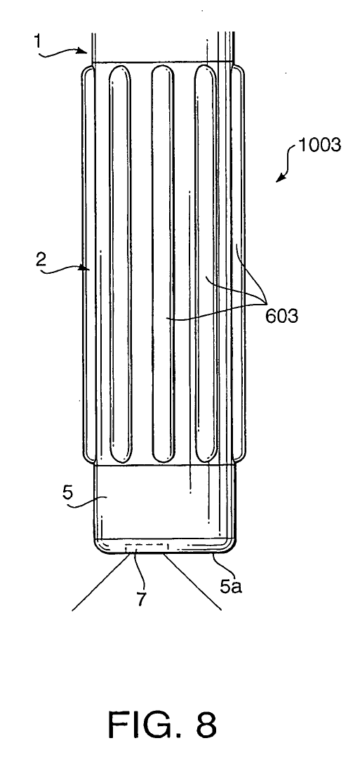

FIG. 8 is a side view of an inserted portion 1 including a bendable portion 2 of an endoscope 1003 according to a third embodiment of the present invention. In the present embodiment, a plurality of elongated protrusions 603, which are spaced from each other, are formed on the entire length of the bendable portion 2 in parallel with the axial direction of the bendable portion 2 when the bendable portion 2 is linearly straightened. With this configuration, the endoscope 1003 is equivalently advantageous to the endoscope 1002 of the second embodiment. It should be noted, however, the protrusions 603 are not necessarily spaced evenly to each other. It should be also noted that at least one of the protrusions 603 may be formed, for example, to be corrugated, in parallel with the axial direction of the bendable portion 2. Further, the number of the protrusion 603 is not limited, as long as at least one protrusion 603 is provided to the bendable portion 2

### Fourth Embodiment

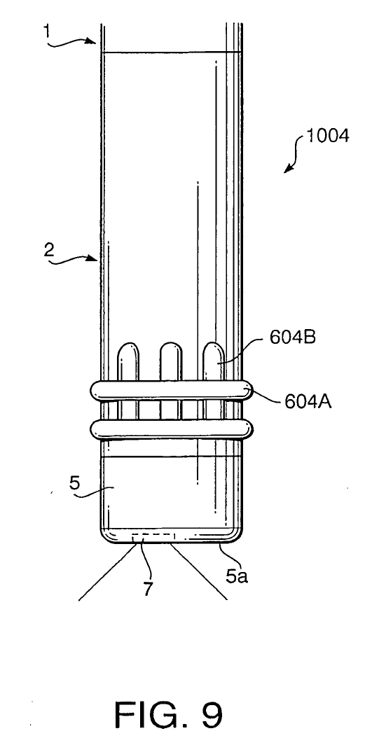

FIG. 9 is a side view of an inserted portion 1 including a bendable portion 2 of an endoscope 1004 according to a fourth embodiment of the present invention. The bendable portion 2 is provided with a plurality of annular protrusions 604A, which are protrusively formed in parallel with the circumferential direction of the bendable portion 2, and a plurality of axial protrusions 604B, which are protrusively formed in parallel with the axial direction of the bendable portion 2 when the bendable portion 2 is linearly straightened. It should be noted that the annular protrusions 604A and the axial protrusions 604B are not necessarily spaced evenly to each other. Further, the number of the annular protrusion 604A and the axial protrusion 604B is not limited, as long as at least one protrusion 604A and at least one protrusion 604A is provided with the bendable portion 2. With this configuration, the endoscope 1004 is advantageous to perform the pulling operation and the twisting operation to the colons, therefore the colons are effectively straightened and the inserted portion 1 can be securely advanced in the colons.

### Fifth Embodiment

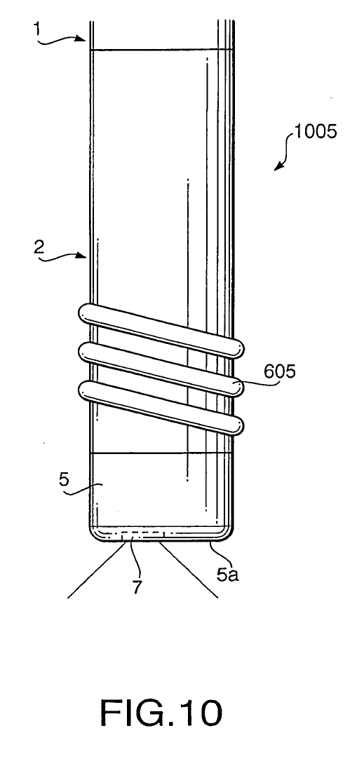

FIG. 10 is a side view of an inserted portion 1 of an endoscope 1005 according to a fifth embodiment of the present invention. In the present embodiment, a line of spiral protrusion 605 is provided on the circumferential surface of the bendable portion 2. The number of the line may be more than one, and a plurality of lines of spiral protrusions 605 may intersect with each other on the circumferential surface of the bendable portion 2.

### Sixth Embodiment

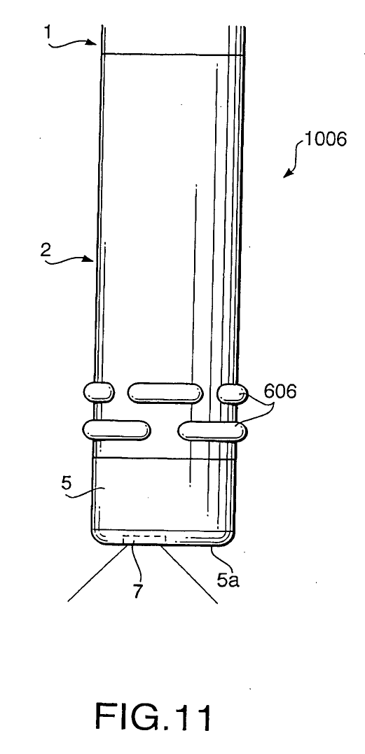

FIG. 11 is a side view of an inserted portion 1 of an endoscope 1006 according to a sixth embodiment of the present invention. In the present embodiment, a plurality of partially broken annular protrusions 606 are formed on the circumferential surface of the bendable portion 2. The protrusions 606 may be arranged regularly or irregularly. It should be noted that protrusions 606 may be in various forms, and for example, dotted protrusions may be arranged regularly or irregularly along the circumferential surface of the bendable portion 2.

### Seventh Embodiment

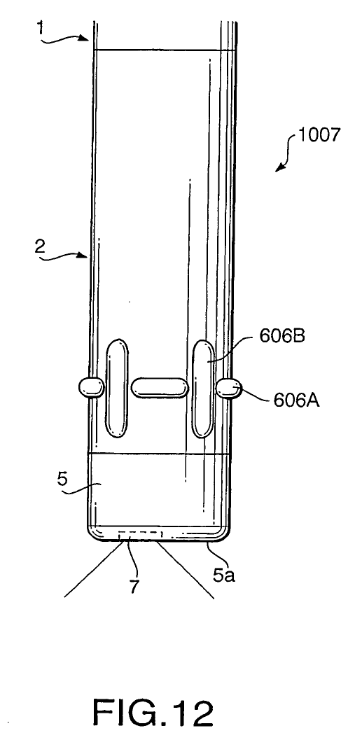

FIG. 12 is a side view of an inserted portion 1 of an endoscope 1007 according to a seventh embodiment of the present invention. In the present embodiment, a plurality of partially broken annular protrusion 606A are provided on the circumferential surface of the bendable portion 2, and a plurality of axial protrusions 606B are provided between the protrusions 606A. The axial protrusions 606B are elongated in parallel with the axial direction of the bendable portion 2 when the bendable portion 2 is linearly straightened, and perpendicularly to the protrusions 606A. The protrusions 606A and 606B may be arranged regularly or irregularly.

### Eighth Embodiment

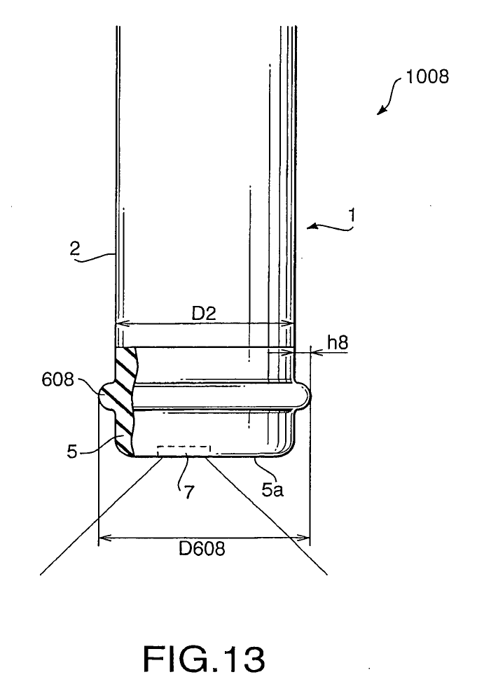

FIG. 13 is a side view of an inserted portion 1 of an endoscope 1008 according to an eighth embodiment of the present invention. On an outer peripheral surface of a distal unit 5, a flange 608 is protrusively and annularly formed in parallel with a circumferential direction of the distal unit 5.

An outer diameter D608 of the flange 608 is configured to be greater than an outer diameter D2 of the bendable portion 2 (more specifically, the diameter of the bendable portion 2 at the closest to the distal unit 5). The flange 608 is formed integrally with the circumferential surface of the distal unit 5 and to have a cross-sectional shape of an approximate semicircle, of which the edge is smoothly curved and continued to the circumferential surface of the distal unit 5. A height h8 of the flange 608 is formed to be in a range for example from 1 mm to 3 mm. It should be noted that the cross-sectional shape of the flange 608 is not limited to a semicircle, but may be in other shapes, including for example an approximate triangle and an approximate square.

In the above-described configuration, the distal unit 5 with the flange 608 of the endoscope of the present invention is firmly hooked to the crease 100, and the distal unit 5 is less likely to be detached from the crease 100. Therefore, the colons are effectively straightened and the inserted portion 1 can be securely advanced in the colons.

### Ninth Embodiment

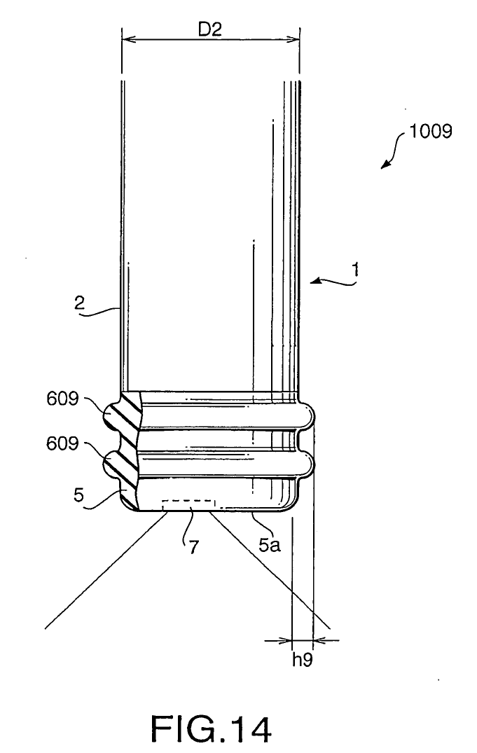

FIG. 14 is a side view of an inserted portion 1 of an endoscope 1009 according to a ninth embodiment of the present invention. On an outer peripheral surface of a distal unit 5, a plurality of flanges 609, which are spaced from each other, are formed in parallel with the circumferential direction of the distal unit 5. With this configuration, the plurality of flanges 609 are more firmly hooked to the crease 100, therefore, a height h9 of the flanges 609 can be formed to be smaller than the flange 608 of the eighth embodiment as shown in FIG. 13, for example in a range from 0.5 mm to 2 mm.

### Tenth Embodiment

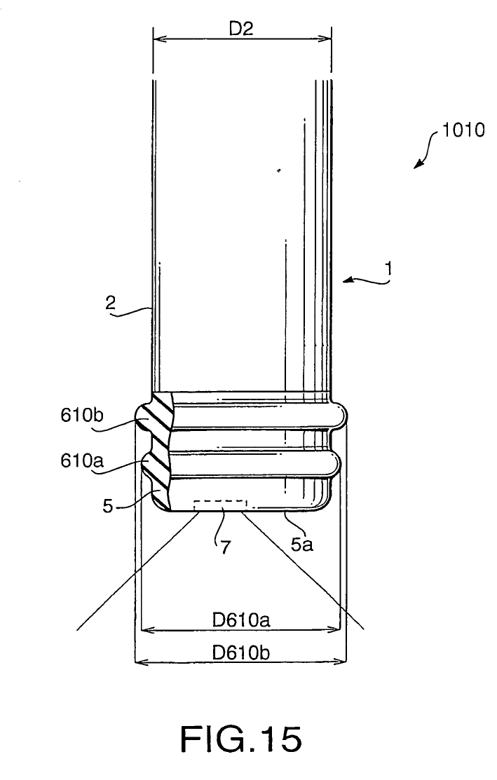

FIG. 15 is a side view of an inserted portion 1 of an endoscope 1010 according to a tenth embodiment of the present invention. On an outer peripheral surface of a distal unit 5, a plurality of flanges, which are spaced from each other, are formed in parallel with the circumferential direction of the distal unit 5.

It should be noted that, in the present embodiment, an outer diameter D610a of a first flange of the plurality of flanges that is closer to the distal end of the distal unit 5 (i.e., the flange 610a in FIG. 15) is formed to be smaller than an outer diameter D610b of a second flange that is closer to the proximal end of the distal unit 5 (i.e., the flange 610b in FIG. 15). With this configuration, the distal unit 5 is less likely to be interfered in the colons unintentionally with the crease 100 when the inserted portion 1 is inserted into the colons.

### Eleventh Embodiment

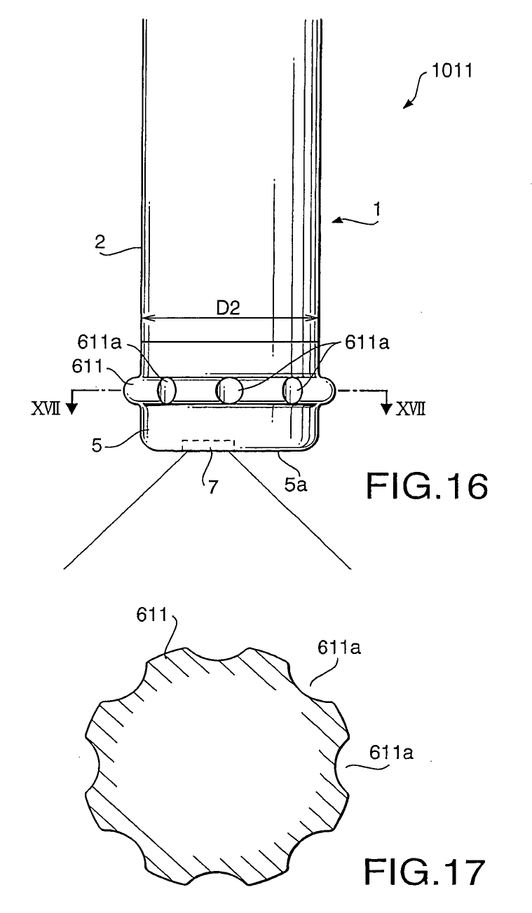

FIG. 16 is a side view of an inserted portion 1 of an endoscope 1011 according to an eleventh embodiment of the present invention. On an outer peripheral surface of a distal unit 5, a flange 611 is formed in parallel with the circumferential direction of the distal unit 5. FIG. 17 is a cross-sectional view of the flange 611 taken along the line XVII-XVII in FIG. 16. The flange 611 is provided with a plurality of recessed portions 611a, which are spaced from each other and arranged in parallel with the circumferential direction of the flange 611.

In the above-described configuration, the distal unit 5 with the flange 611 including the recessed portions 611a of the endoscope of the present invention is firmly hooked to the crease 100, and the distal unit 5 is less likely to be detached from the crease 100. Therefore, the colons are effectively straightened and the inserted portion 1 can be securely advanced in the colons. It should be also noted that the number of the recessed portions 611a is not limited, but is preferable to be in a range from three to twelve for practical reasons.

### Twelfth Embodiment

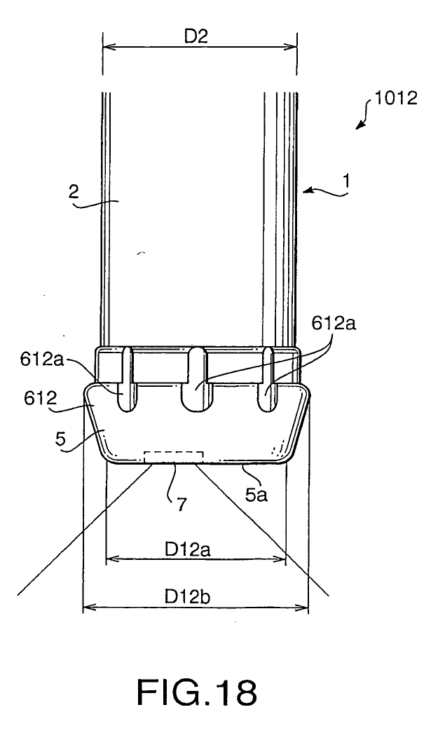

FIG. 18 is a side view of an inserted portion 1 of an endoscope 1012 according to a twelfth embodiment of the present invention. A flange 612 in the present embodiment is configured to be tapered with an outer diameter D12a at an distal end of the distal unit 5 being smaller than an outer diameter D12b at a boundary between the distal unit 5 and an bendable portion 2. With this configuration, inserting the distal unit 5 in the colons can be conducted smoothly. A plurality of recessed portions 612a may be formed on the flange 612 and continuously on the circumferential surface of the distal unit 5.

### Thirteenth Embodiment

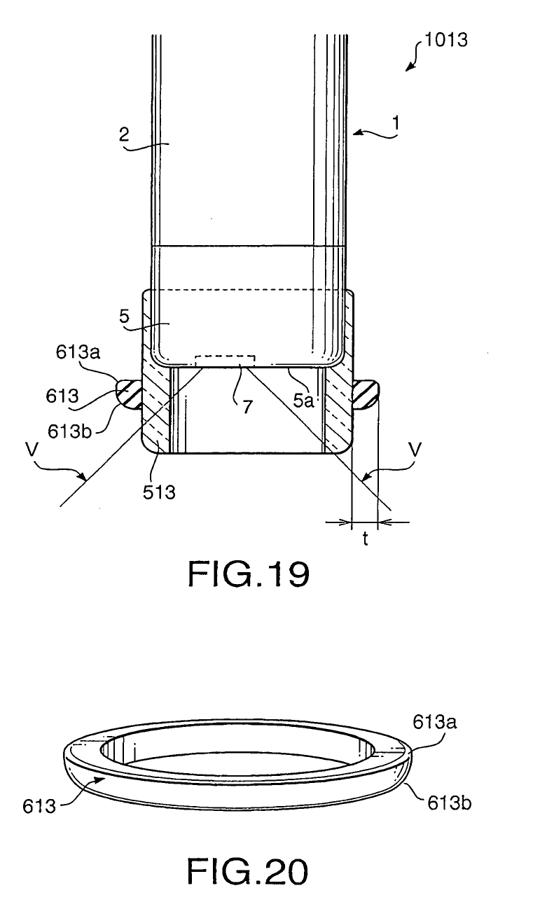

FIG. 19 is a cross-sectional side view of an inserted portion 1 of an endoscope 1013 according to a thirteenth embodiment of the present invention. A cylindrical hood 513 is configured to protrude forwardly from a distal end of the distal unit 5, and to be detachably attached to a distal unit 5. The hood 513 may be attached to the distal unit 5 by being elastically tightened, engaged by an engaging structure including a pair of an engaging protrusion and a mating recessed portion, screwed to the distal unit 5, or in other known configurations. However, the hood 513 may be configured to be undetachably fixed to the distal unit 5.

A ring-like flange 613 is provided on an outer peripheral surface of the hood 513 in parallel with the circumferential direction of the hood 513. FIG. 20 is a perspective view of the flange 613 of the endoscope 1013 according to the thirteenth embodiment of the present invention.

The hood 513 is formed with a transparent material, in which case a scope for observation, which is indicated by V, V in FIG. 19, is not intercepted by the hood 513.

The flange 613 is formed to have a thickness t, which is in a range for example from 1 mm to 3 mm, and is fixed to the outer peripheral surface of the hood 513 for example with an adhesive material.

The flange 613 includes a proximal edge 613a, which is closer the proximal end of the hood 513 when the flange 613 is fixed to the hood 513, and a distal edge 613b, which is closer to the distal end of the hood 513 when the flange 613 is fixed to the hood 513. The distal edge 613b is chamfered smoothly so that the distal edge 613b is not unintentionally interfered with the crease 100 when the inserted portion 1 is inserted into the colons. The proximal edge 613a is also curved, however at a smaller curvature than the curvature of the distal edge 613b, so that the proximal edge 613a can be hooked firmly enough to the crease 100 when the inserted portion 1 is pulled toward the anus.

It should be noted that the flange 613 may be formed integrally with the hood 513 in the same material as the hood 513.

### Fourteenth Embodiment

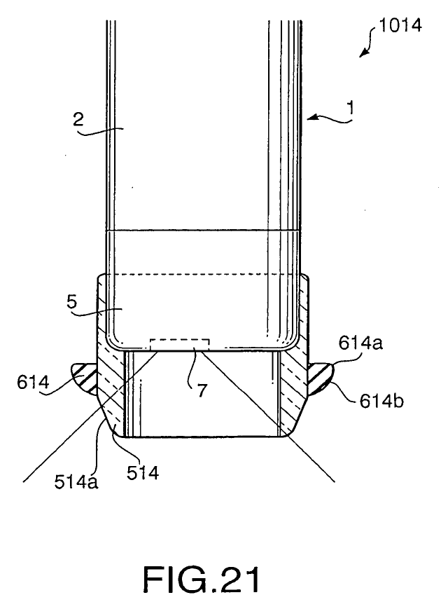

FIG. 21 is a cross-sectional side view of an inserted portion 1 of an endoscope 1014 according to a fourteenth embodiment of the present invention. A hood 514 includes a tapered portion 514a, which is tapered from a position where the flange 614 is fixed to toward a distal end of the hood 514. An distal edge 614b of the flange 614 is chamfered smoothly at a greater curvature than the curvature of the distal edge 613b of the thirteenth embodiment shown in FIG. 20. With this configuration, the distal edge 614b is not unintentionally interfered with the crease 100 when the inserted portion 1 is inserted into the colons.

### Fifteenth Embodiment

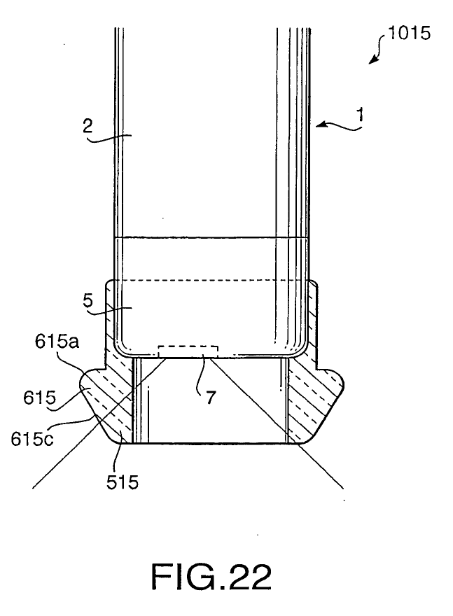

FIG. 22 is a cross-sectional side view of an inserted portion 1 of an endoscope 1015 according to a fifteenth embodiment of the present invention. A flange 615 is integrally formed with a hood 515. A peripheral surface 615c of the flange 615 is formed to be tapered toward a distal end of the hood 515, so that the hood 515 can be smoothly inserted into the colons.

### Sixteenth Embodiment

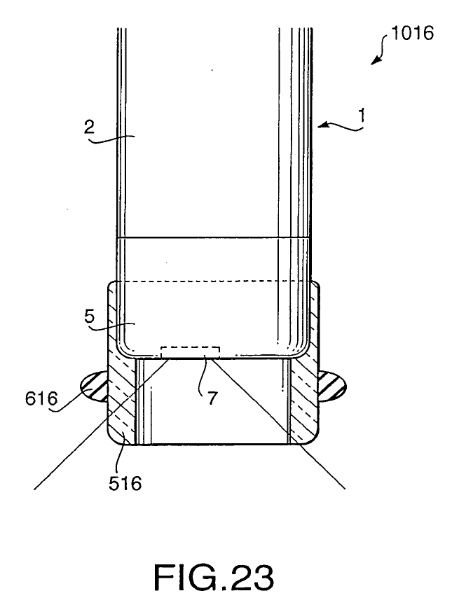

FIG. 23 is a cross-sectional side view of an inserted portion 1 of an endoscope 1016 according to a sixteenth embodiment of the present invention. A flange 616 is formed to have a cross-sectional shape of a semicircle. The remaining configuration of the endoscope 1016 is similar to the configuration of the endoscope 1013, which is described in the thirteenth embodiment. With this configuration, the flange 616 may be hooked less firmly to the crease 100 than the flange 613 of the endoscope 613 is hooked to the crease 100, however, the flange 616 is still advantageous in being hooked to the crease 100 so that the colons can be partially pulled toward the proximal end of the endoscope 1016.

### Seventeenth Embodiment

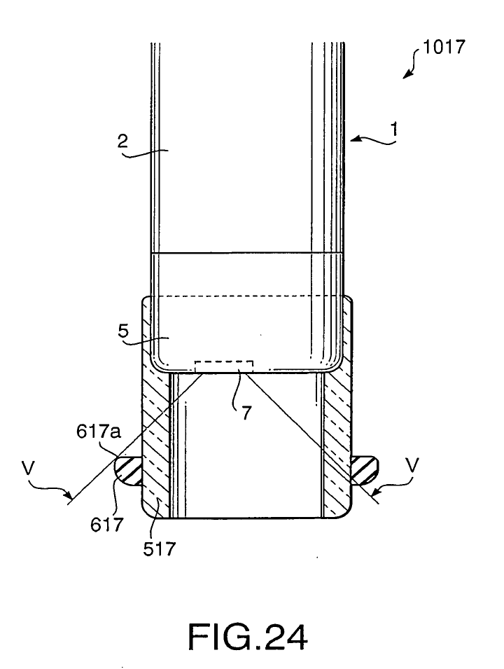

FIG. 24 is a cross-sectional side view of an inserted portion 1 of an endoscope 1017 according to a seventeenth embodiment of the present invention. A hood 517 is formed to be longer in the axial direction to protrude forwardly than the hood 513 of the thirteenth embodiment shown in the FIG. 19. With this configuration, a flange 617 can be in a scope for observation, which is indicated by V, V. Thus, the operator can visually recognize the flange 617 to be hooked to the crease 100.

### Eighteenth Embodiment

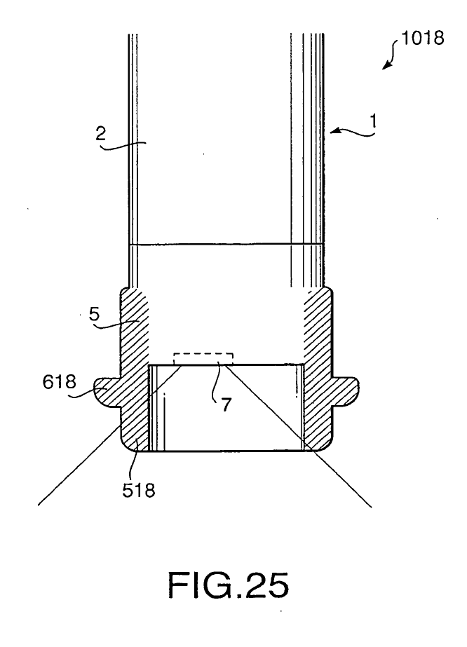

FIG. 25 is a cross-sectional side view of an inserted portion 1 of an endoscope 1018 according to an eighteenth embodiment of the present invention. A hood 518 is made in an opaque material and integrally formed with a distal unit 5. Also with this configuration, the endoscope 1018 is advantageous in being hooked to the crease 100 to have the colons partially twisted and/or pulled.

### Nineteenth Embodiment

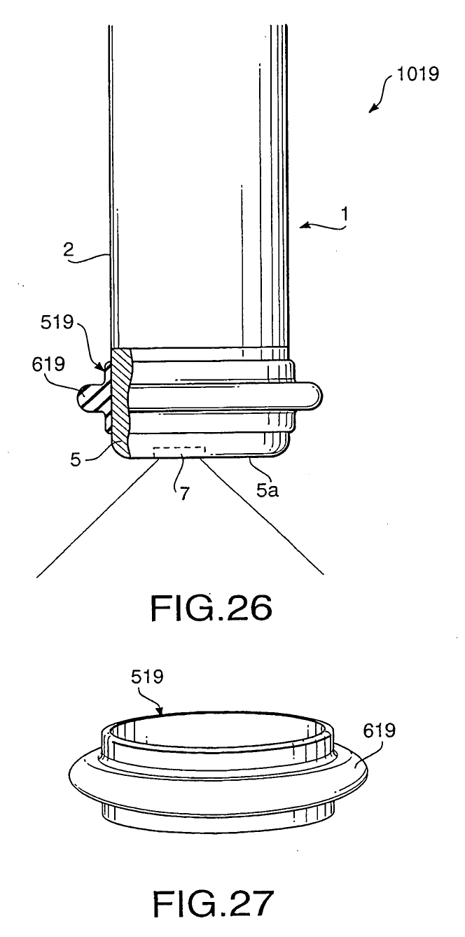

FIG. 26 is a side view of an inserted portion 1 of an endoscope 1019 according to a nineteenth embodiment of the present invention. On an outer peripheral surface of a distal unit 5, a support band 519 that encircles the distal unit in parallel with a circumferential direction of the distal unit 5 is detachably attached. The support band 519 is integrally formed with a flange 619, which is formed to protrude outwardly and annularly in parallel with the circumferential direction of the flange 619.

FIG. 27 is a perspective view of the support band 519 of the endoscope 1019 according to the nineteenth embodiment of the present invention. The support band 519 is made of for example in an elastic material, such as rubber or plastics. The support band 519 is formed to have an inner diameter that is smaller than an outer diameter of the distal unit 5. The support band 519 is attached to the distal unit 5 by being elastically expanded outwardly.

The flange 619 is formed to have a cross-sectional shape of an approximate semicircle, of which the edge is smoothly curved and continued to the circumferential surface of the support band 519. A height h19 of the flange 619 is formed to be in a range for example from 1 mm to 3 mm. It should be noted that the cross-sectional shape of the flange 619 is not limited to a semicircle, but-may be in other shapes, including for example an approximate triangle and an approximate square.

The above-configured support band 519 is attached to the distal unit by being elastically tightened around the circumferential surface of the distal unit 5, so that the support band 519 does not slip to protrude forwardly toward the distal end of the distal unit and slip off the distal unit 5.

### Twentieth Embodiment

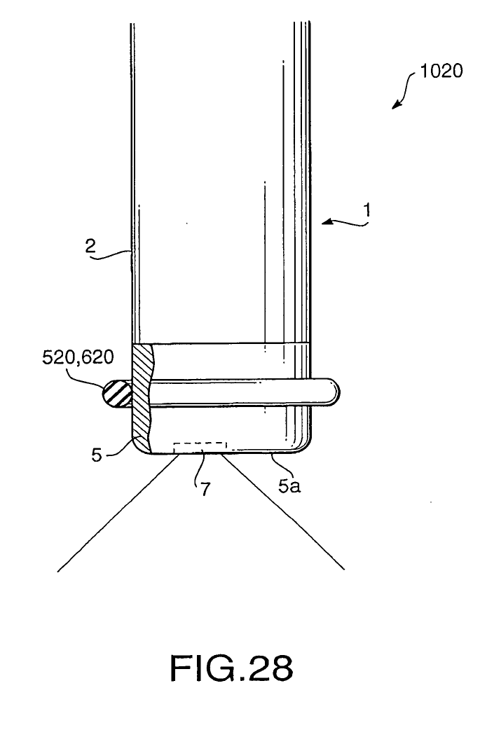

FIG. 28 is a side view of an inserted portion 1 of an endoscope 1020 according to a twentieth embodiment of the present invention. A support band 520 is attached on an outer peripheral surface of a distal unit 5. A flange 620 is formed to be completely included in the support band 520, therefore the entire support band 520 appears to coincide with the flange 620. In the present embodiment, the flange 620 is formed to have a cross-sectional shape of an approximate circle. With this configuration, the endoscope 1020 is equivalently advantageous to the endoscope 1019 of the nineteenth embodiment.

### Twenty-First Embodiment

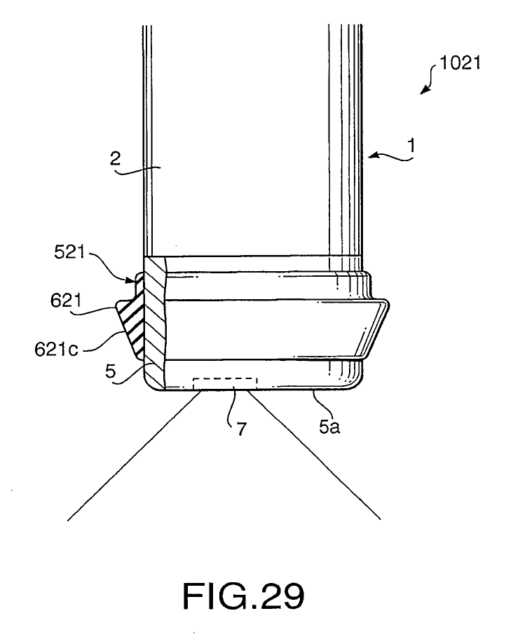

FIG. 29 is a side view of an inserted portion 1 of an endoscope 1021 according to a twenty-first embodiment of the present invention. A circumferential surface 621c of a flange 621 is configured to be tapered toward a distal end of the flange 621, so that a distal unit 5 including the flange 621 can be smoothly inserted into the colons.

### Twenty-Second Embodiment

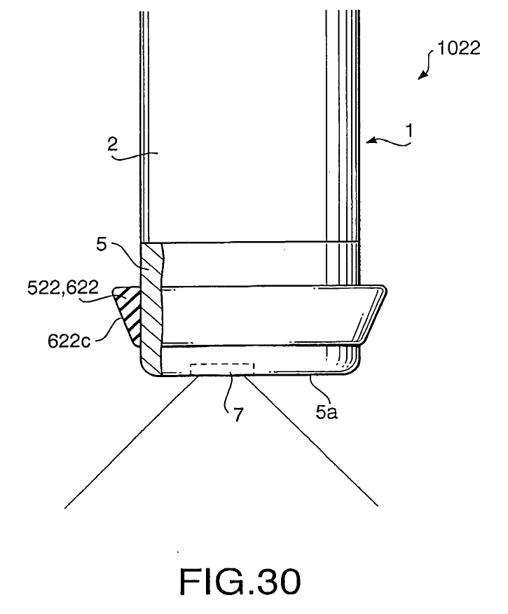

FIG. 30 is a side view of an inserted portion 1 of an endoscope 1022 according to a twenty-second embodiment of the present invention. A flange 622 is formed to be included in the support band 522, therefore the entire support band 522 appears to coincide with the flange 620. A circumferential surface 622c of the flange 621 is configured to be tapered toward a distal end of the flange 622, so that a distal unit 5 including the flange 622 can be smoothly inserted into the colons. With this configuration, the endoscope 1022 is equivalently advantageous to the endoscope 1021 of the twenty-fist embodiment.

### Twenty-Third Embodiment

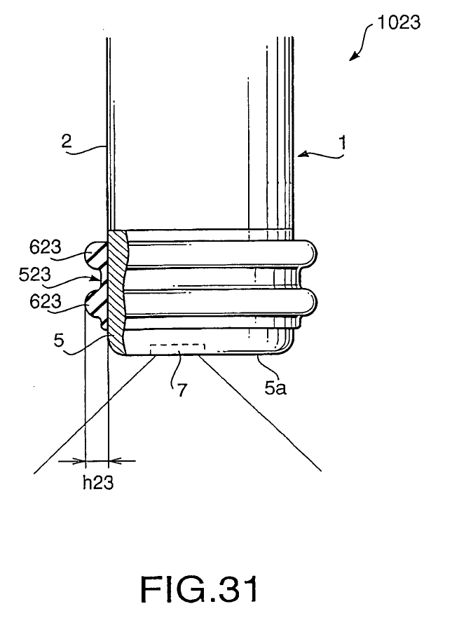

FIG. 31 is a side view of an inserted portion 1 of an endoscope 1023 according to a twenty-third embodiment of the present invention. A support band 523 is provided with a plurality of flanges 623, which are spaced from each other and formed in parallel with the circumferential direction of a distal unit 5. Each of the plurality of flanges 623 has a cross-sectional shape of an approximate circle. With this configuration, the plurality of flanges 623 are more firmly hooked to the crease 100, therefore, a height h23 of the flanges 623 can be formed to be smaller than for example the flange 620 of the twentieth embodiment as shown in FIG. 28, for example in a range from 0.5 mm to 2 mm.

### Twenty-Fourth Embodiment

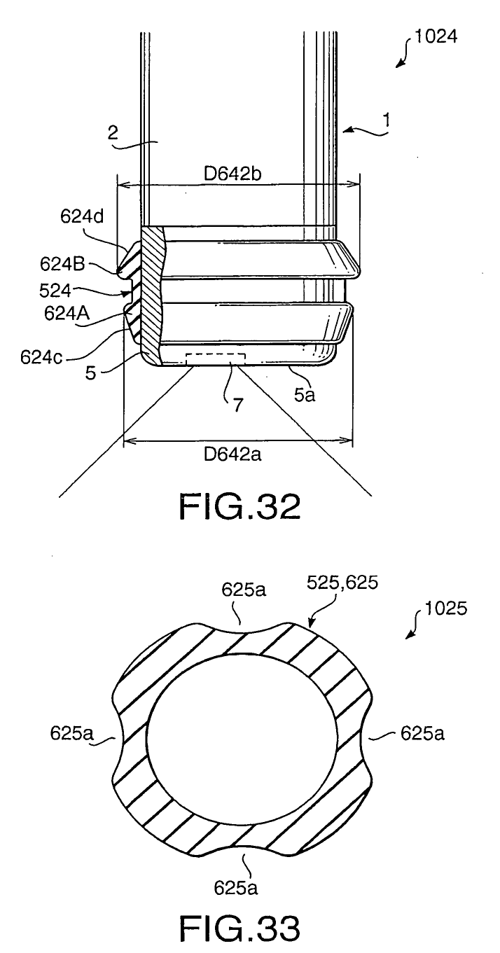

FIG. 32 is a side view of an inserted portion 1 of an endoscope 1024 according to a twenty-fourth embodiment of the present invention. A support band 524 is provided with a plurality of flanges 624A, 624B, which are spaced from each other and formed in parallel with the circumferential direction of a distal unit 5. An circumferential surface 624c of the flange 624A, which is closer to a distal end of the distal unit 5, is configured to be tapered toward the distal end of the distal unit 5, while a circumferential surface 624d of the flange 624B, which is closer to a proximal end of the distal unit 5, is configured to be tapered toward the proximal end of the distal unit 5. With this configuration, the flanges 624A, 624B are not interfered with the crease 100 when the inserted portion 1 is advanced or retracted quickly inside the colons, while the flanges 624A, 624B are hooked to the crease 100 when the inserted portion is steadily advanced or retracted.

It should be noted that an outer diameter D624a of the flange 624A is formed to be smaller than an outer diameter D624b of the flange 624B, so that the flanges 624A, 624B are not likely to be interfered with the crease 100 when the inserted portion 1 is retracted from the colons.

### Twenty-Fifth Embodiment

FIG. 33 is a cross-sectional view of a support band 525 with a flange 625 of an endoscope 1025 taken along a line that is perpendicular to an axial direction of the support band 525 according to a twenty-fifth embodiment of the present invention. The flange 625 is provided with a plurality of recessed portions 625a, which are spaced from each other and arranged in parallel with the circumferential direction of the flange 625. Such a plurality of recessed portions 625a are applicable to the flanges 619, 620, 621, 623, and 624 in the previous embodiments. It should be noted that the number of the recessed portion 625a is not limited, but is preferable to be in a range from three to twelve for practical reasons. With the above-described configuration, each of the flanges 619, 620, 621, 623, and 624 in the previous embodiments may be hooked firmly to the crease 100, so that the looped colon is turned along with the inserted portion 1 to be partially straightened and the inserted portion 1 can be effectively advanced in the colon.

### Twenty-Sixth Embodiment

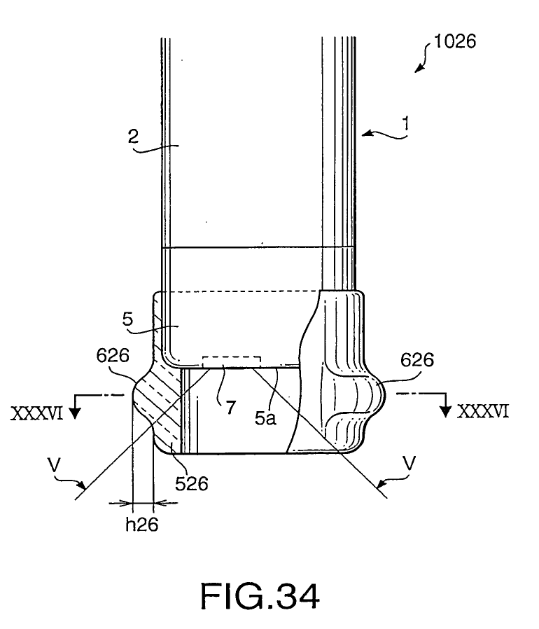

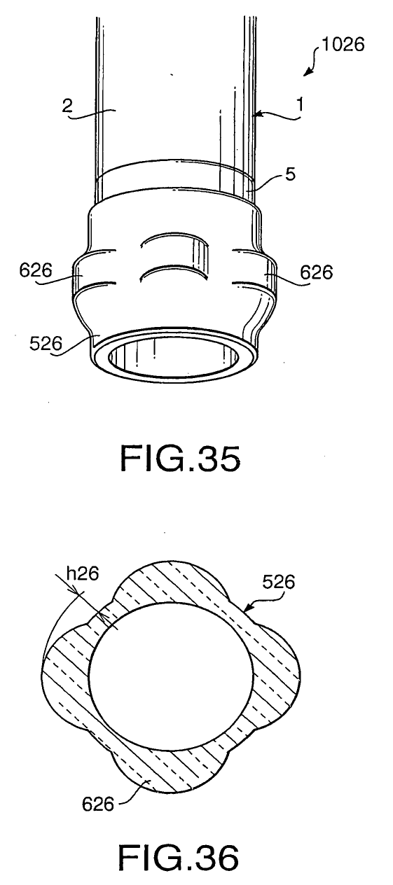

FIG. 34 is a side view of an inserted portion 1 of an endoscope 1026 according to a twenty-sixth embodiment of the present invention. FIG. 35 is a perspective side view of the inserted portion 1 of the endoscope 1026 according to the twenty-sixth embodiment of the present invention. A hood 526 is configured to protrude forwardly from a distal end of a distal unit 5, and to be detachably attached to a distal unit 5. On an outer peripheral surface of the hood 526, a plurality of bosses 626 are integrally formed with the hood 526. The bosses 626 are evenly spaced from each other, and arranged in a line that is parallel with a circumferential direction of the hood 526. FIG. 36 is a cross-sectional view of the hood 526 taken along the line XXXVI-XXXVI in FIG. 34.

The hood 526 may be attached to the distal unit 5 by being elastically tightened, engaged by an engaging structure including a pair of an engaging protrusion and a mating recessed portion, screwed to the distal unit 5, or in other known configurations. However, the hood 526 may be configured to be undetachably fixed to the distal unit 5.

The hood 526 is formed with a transparent material, in which case a scope for observation, which is indicated by V, V in FIG. 34, is not intercepted by the hood 526.

Each of the bosses 626 is formed to protrude outwardly from the outer peripheral surface of the hood 526, and a height h26 of the boss 626 is formed to be in a range for example from 1 mm to 3 mm. In the present embodiment, as shown in FIG. 36, four bosses 626 are arranged in a line parallel with the circumferential direction of the hood 526 at an angle, for example 90 degrees, to each other.

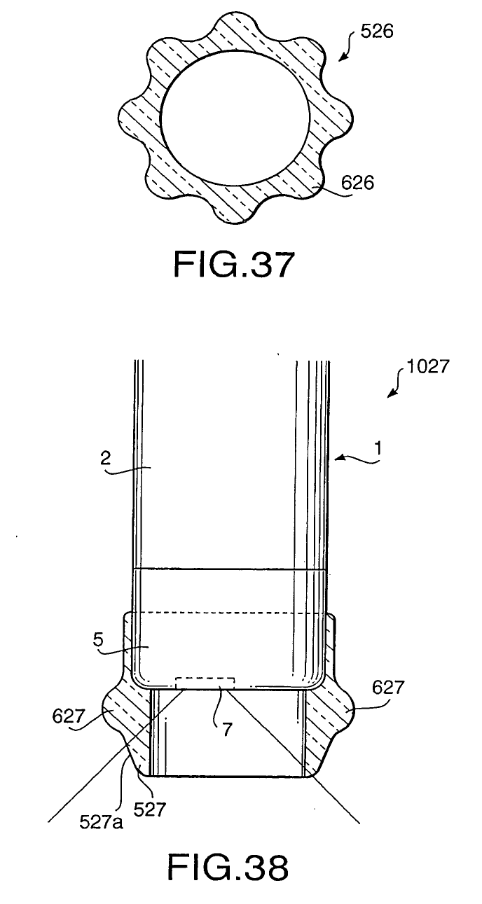

It should be noted that the bosses may not be arranged evenly spaced to each other, and may not be necessarily in a line that is parallel with the circumferential direction. The number of the bosses 626 is not limited to four, but may be for example eight as shown in FIG. 37. However, it should be noted that the number of the bosses 626 is preferable to be in a range from three to twelve for practical reasons.

### Twenty-Seventh Embodiment

FIG. 38 is a cross-sectional side view of an inserted portion 1 of an endoscope 1027 according to a twenty-seventh embodiment of the present invention. A hood 527 includes a tapered portion 527a, which is tapered from a position where the flange 627 is provided to toward a distal end of the hood 527. With this configuration, inserting the distal unit 5 in the colons can be conducted smoothly. On an outer peripheral surface of the hood 527, a plurality of bosses 627 are integrally formed with the hood 527.

### Twenty-Eighth Embodiment

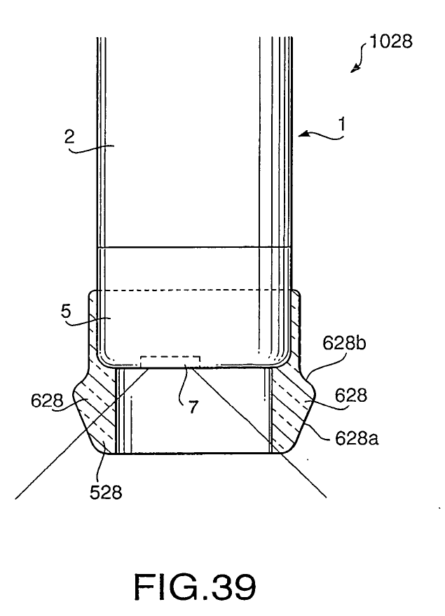

FIG. 39 is a cross-sectional side view of an inserted portion 1 of an endoscope 1028 according to a twenty-eighth embodiment of the present invention. On an outer peripheral surface of the hood 528, a plurality of bosses 628 are integrally formed with the hood 528. Each of the bosses 628 includes a distal peripheral portion 628a, which is formed to be tapered from a most bossed portion of the boss 628 toward a distal end of the hood 528, so that the hood 528 can be smoothly inserted into the colons. Each of the bosses 628 further includes a proximal peripheral portion 628b, which is formed to protrude from the outer peripheral surface of the hood 528.

With this configuration, it should be noted that the proximal peripheral portions 628b can be effectively hooked to the crease 100 when the inserted portion is pulled toward the proximal end of the endoscope 1028 to partially shorten the colons, and the distal peripheral portions 628a can be effectively hooked to the crease 100 when the inserted portion 1 is twisted in the circumferential direction to partially straighten the colons.

### Twenty-Ninth Embodiment

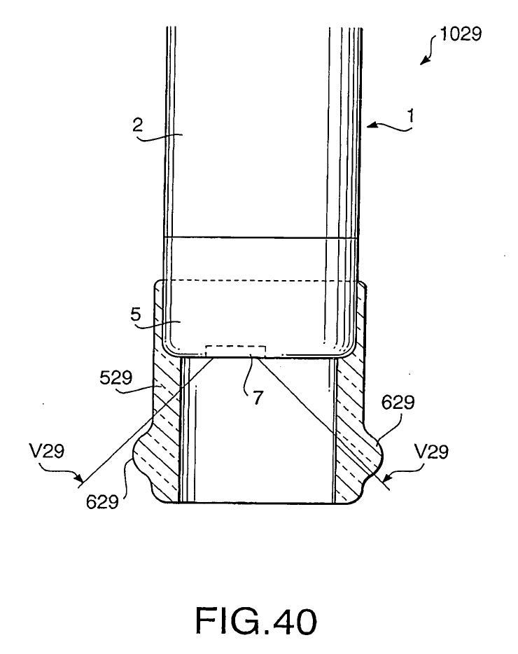

FIG. 40 is a cross-sectional side view of an inserted portion 1 of an endoscope 1029 according to a twenty-ninth embodiment of the present invention. On an outer peripheral surface of a hood 529, a plurality of bosses 629 are integrally formed with the hood 529. The hood 529 is formed to be longer in the axial direction to protrude forwardly than the hood 526 of the twenty-sixth embodiment shown in the FIG. 34. With this configuration, the bosses 629 can be in a scope for observation, which is indicated by V29, V29. Thus, the operator can visually recognize the bosses 629 to be hooked to the crease 100.

### Thirtieth Embodiment

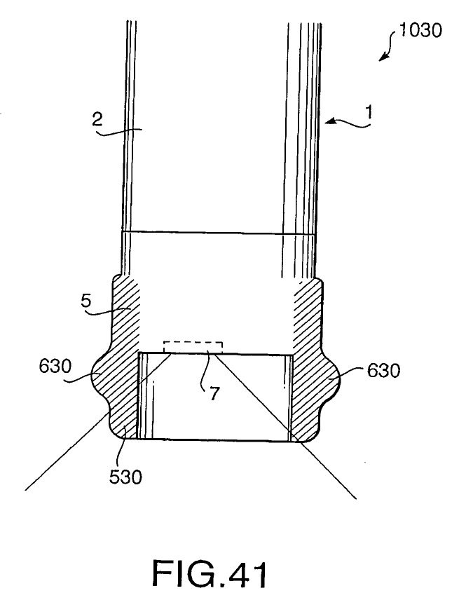

FIG. 41 is a cross-sectional side view of an inserted portion 1 of an endoscope 1030 according to a thirtieth embodiment of the present invention. A hood 530 is made in an opaque material and integrally formed with a distal unit 5. On an outer peripheral surface of the hood 530, a plurality of bosses 630 are integrally formed with the hood 530. Also with this configuration, the endoscope 1030 is advantageous in being hooked to the crease 100 to have the colons partially twisted and/or pulled.

The present disclosure relates to the subject matter contained in Japanese Patent Application Nos. 2005-006975, 2005-006976, 2005-006977, 2005-006978, and 2005-006980, filed on Jan. 14, 2005, which is expressly incorporated herein by reference in their entireties.

What is claimed is:

1. An endoscope comprising:
an inserted portion, which is to be inserted into a tubular organ, including a bendable portion that is adapted to bend arbitrarily by a remote operation and a distal unit with a window for observation to capture an image of inside of the tubular organ,
wherein at least one protrusion is formed on a outer peripheral surface of the bendable portion.

2. The endoscope according to claim 1,
wherein the bendable portion is coated with a flexible tubular member, and,
wherein the at least one protrusion is integrally formed with the tubular member.

3. The endoscope according to claim 2, wherein the at least one protrusion is protrusively formed in parallel with a circumferential direction of the bendable portion.

4. The endoscope according to claim 3, wherein a plurality of the at least one protrusion are spaced from each other in an axial direction of the bendable portion.

5. The endoscope according to claim 2, wherein the at least one protrusion is protrusively formed in parallel with an axial direction of the bendable portion.

6. The endoscope according to claim 5, wherein a plurality of the at least one protrusion are spaced from each other in parallel with a circumferential direction of the bendable portion.

7. The endoscope according to claim 2,
wherein at least one protrusion is formed in parallel with an axial direction of the bendable portion, and
wherein at least one protrusion is formed in parallel with a circumferential direction of the bendable portion.

8. The endoscope according to claim 2, wherein the at least one protrusion is formed to be in a line of a spiral.

9. The endoscope according to claim 1, wherein the at least one protrusion is protrusively formed in parallel with a circumferential direction of the bendable portion.

10. The endoscope according to claim 9, wherein a plurality of the at least one protrusion are spaced from each other in an axial direction of the bendable portion.

11. The endoscope according to claim 1, wherein the at least one protrusion is protrusively formed in parallel with an axial direction of the bendable portion.

12. The endoscope according to claim 11, wherein a plurality of the at least one protrusion are spaced from each other in parallel with a circumferential direction of the bendable portion.

13. The endoscope according to claim 1,
wherein at least one protrusion is formed in parallel with an axial direction of the bendable portion, and
wherein at least one protrusion is formed in parallel with a circumferential direction of the bendable portion.

14. The endoscope according to claim 1, wherein the at least one protrusion is formed to be in a line of a spiral.

15. The endoscope according to claim 1, wherein a plurality of the at least one protrusion are formed to be in a broken line.

16. The endoscope according to claim 1, wherein the at least one protrusion is formed in a vicinity of a distal end of the bendable portion.

17. The endoscope according to claim 1, wherein the at least one protrusion is formed on an entire length of the bendable portion.

18. An endoscope comprising:
an inserted portion, which is to be inserted into a tubular organ, including a bendable portion that is adapted to bend arbitrarily by a remote operation and a distal unit with a window for observation to capture an image of inside of the tubular organ,
wherein at least one flange with an outer diameter being greater than an outer diameter of the bendable portion is protrusively formed on an outer peripheral surface of the distal unit.

19. The endoscope according to claim 18, wherein a plurality of the at least one flange are spaced from each other on the outer peripheral surface of the distal unit.

20. The endoscope according to claim 19, wherein one of the plurality of the at least one flange that is closer to a distal end of the distal unit is formed to have an outer diameter that is smaller than an outer diameter of another one of the plurality of the at least one flange that is closer to a proximal end of the distal unit.

21. The endoscope according to claim 20, wherein a plurality of recessed portions are formed on the at least one flange and spaced from each other in parallel with a circumferential direction of the at least one flange.

22. The endoscope according to claim 21, wherein the at least one flange is formed to be tapered toward the distal end of the distal unit.

23. The endoscope according to claim 20, wherein the at least one flange is formed to be tapered toward the distal end of the distal unit.

24. The endoscope according to claim 19, wherein a plurality of recessed portions are formed on the at least one flange and spaced from each other in parallel with a circumferential direction of the at least one flange.

25. The endoscope according to claim 24, wherein the at least one flange is formed to be tapered toward the distal end of the distal unit.

26. The endoscope according to claim 19, wherein the at least one flange is formed to be tapered toward the distal end of the distal unit.

27. The endoscope according to claim 18, wherein a plurality of recessed portions are formed on the at least one flange and spaced from each other in parallel with a circumferential direction of the at least one flange.

28. The endoscope according to claim 27, wherein the at least one flange is formed to be tapered toward the distal end of the distal unit.

29. The endoscope according to claim 18, wherein the at least one flange is formed to be tapered toward the distal end of the distal unit.

30. An endoscope comprising:
an inserted portion, which is to be inserted into a tubular organ, including a bendable portion that is adapted to bend arbitrarily by a remote operation and a distal unit with a window for observation to capture an image of inside of the tubular organ, and
a cylindrical hood that is provided to a distal end of the distal unit and formed to protrude forwardly from the distal end of the distal unit,
wherein at least one flange that encircles the cylindrical hood in a circumferential direction of the cylindrical hood is attached to an outer peripheral surface of the cylindrical hood.

31. The endoscope according to claim 30, wherein the cylindrical hood is made of a transparent material.

32. The endoscope according to claim 31, wherein the cylindrical hood is configured to be detachable from the distal unit.

33. The endoscope according to claim 30, wherein the cylindrical hood is configured to be detachable from the distal unit.

34. The endoscope according to claim 30, wherein the at least one flange is formed integrally with the cylindrical hood.

35. The endoscope according to claim 30,
wherein the at least one flange includes a proximal edge that is closer to a proximal end of the cylindrical hood and a distal edge that is closer to a distal end of the cylindrical hood, the distal edge being chamfered in a greater curvature than a curvature of the proximal edge being chamfered.

36. The endoscope according to claim 30, wherein the cylindrical hood includes a tapered portion that is tapered from a position where the at least one flange is fixed to toward a distal end of the cylindrical hood.

37. The endoscope according to claim 30, wherein an outer peripheral surface of the at least one hood is formed to be tapered toward a distal end of the cylindrical hood.

38. An endoscope comprising:
an inserted portion, which is to be inserted into a tubular organ, including a bendable portion that is adapted to bend arbitrarily by a remote operation and a distal unit with a window for observation to capture an image of inside of the tubular organ,
wherein the distal unit is detachably attached with a support band that encircles the distal unit in parallel with a circumferential direction of the distal unit, and
wherein the support band is integrally formed with at least one flange that protrudes outwardly and annularly in parallel with the circumferential direction of the support band.

39. The endoscope according to claim 38, wherein the support band is configured to be attached to the distal unit so that the support band remain in an attached position.

40. The endoscope according to claim 39, wherein the support band is attached to the distal unit by being elastically tightened around an outer peripheral surface of the distal unit.

41. The endoscope according to claim 38, wherein the support band is attached to the distal unit by being elastically tightened around an outer peripheral surface of the distal unit.

42. The endoscope according to claim 38, wherein the at least one flange is formed to be >completely included in the support band.

43. The endoscope according to claim 38, wherein the at least one flange is formed to be tapered toward a distal end of the distal unit.

44. The endoscope according to claim 38, wherein a plurality of the at least one flange are formed to be spaced from each other in parallel with a circumferential direction of the support band.

45. The endoscope according to claim 44, wherein one of the plurality of the at least one flange that is closer to a proximal end of the distal unit is formed to be tapered toward the proximal end of the distal unit.

46. The endoscope according to claim 45, wherein a maximum outer diameter of one of the plurality of the at least one flange that is closer to a distal end of the distal unit is formed to be smaller than a maximum outer diameter of the one of the plurality of the at least one flange that is closer to the proximal end of the distal unit.

47. The endoscope according to claim 44, wherein a maximum outer diameter of one of the plurality of the at least one flange that is closer to a distal end of the distal unit is formed to be smaller than a maximum outer diameter of the one of the plurality of the at least one flange that is closer to the proximal end of the distal unit.

48. The endoscope according to claim 38, wherein a plurality of recessed portions are formed on the at least one flange and spaced from each other in parallel with a circumferential direction of the at least one flange.

49. An endoscope comprising:
an inserted portion, which is to be inserted into a tubular organ, including a bendable portion that is adapted to bend arbitrarily by a remote operation and a distal unit with a window for observation to capture an image of inside of the tubular organ, and
a cylindrical hood that is provided to a distal end of the distal unit and formed to protrude forwardly from the distal end of the distal unit,
wherein a plurality of bosses having a rear surface and a peripheral surface that protrude outwardly are formed on an outer peripheral surface of the cylindrical hood and spaced from each other.

50. The endoscope according to claim 49, wherein the cylindrical hood is made of a transparent material.

51. The endoscope according to claim 50, wherein the cylindrical hood is configured to be detachable from the distal unit.

52. The endoscope according to claim 49, wherein the cylindrical hood is configured to be detachable from the distal unit.

53. The endoscope according to claim 49, wherein the plurality of bosses are formed integrally with the cylindrical hood.

54. The endoscope according to claim 49, wherein each of the plurality of bosses includes a tapered portion that is tapered from a position where the each of the plurality of bosses is most protruded outwardly toward a distal end of the cylindrical hood.

55. The endoscope according to claim 49, an outer peripheral surface of the plurality of bosses are formed to be tapered toward a distal end of the cylindrical hood.
